(19) United States
(12) Patent Application Publication
(10) Pub. No.: US 2008/0077225 A1
(43) Pub. Date: Mar. 27, 2008

(54) ACCURACY LUMEN SIZING AND STENT EXPANSION

(76) Inventors: Donald B. Carlin, Pennington, NJ (US); Gerard A. Alphonse, Princeton, NJ (US); Alex Paunescu, Clinton, NJ (US); Fred Rappaport, Jamison, PA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/615,288

(22) Filed: Dec. 22, 2006

Related U.S. Application Data
(60) Provisional application No. 60/826,682, filed on Sep. 22, 2006.

(51) Int. Cl. A61F 2/06

(52) U.S. Cl. 623/1.11

(57) ABSTRACT

The invention relates to a system, method and device for optically determining the shape and size of a lumen of a vessel or body cavity, and of, for example, a balloon stent as it is inflated. The size and shape determination of the lumen of the vessel or body cavity allows for accurate and safe deployment of a stent within the lumen.

### CROSS-REFERENCE TO RELATED APPLICATIONS

This application claims priority to U.S. Provisional Application Ser. No. 60/826,682 entitled “Improved Accuracy Lumen Sizing and Stent Expansion”, filed Sep. 22, 2006, which is incorporated herein by reference in its entirety,

### BACKGROUND

The disclosure contained herein generally relates to a device, system and method for deployment of a stent in a blood vessel. More particularly, the disclosure is related to a device, system and method for in-situ precision measurement of the dimensions of the lumen of a blood vessel and the size of a stent as it is expanded.

Stents are expandable devices inserted into arteries via angioplasty techniques that keep a blood vessel open. They are typically open tubular structures having, for example, struts and ribs that allow expansion when an interiorly placed balloon is inflated. The stents are typically made of metal, although other materials are possible, and are designed to be inflated with sufficient pressure to make close contact with the wall of the lumen in the artery being treated. Manufacturer-recommended pressures for expansion of standard stents are typically in the range of from 4 to 16 atmospheres or higher.

Current clinical practice is to position a balloon and stent in the required location by observation, for example, using angiographic x-ray techniques. The balloon is typically filled with a solution of saline and x-ray contrast agent. The angiogram presents a one-dimensional cross-sectional view of the artery with relatively poor spatial resolution. This is generally the case as the lumen of an artery does not necessarily have a circular cross-section, especially in atherosclerotic sections where it is likely to be an irregular shape. Although the physician may view the artery from more than one angle, the information provided by angiography is limited and insufficient to provide an accurate assessment of the size to which the stent should be or has been inflated.

Angiography is also used in current clinical practice to determine the degree to which an artery is narrowed. Narrowing of the artery is called stenosis and is illustrated in FIG. 1A. Stenosis is caused by the buildup, of plaque (14) commonly referred to as lesions on an interior area of an artery wall (10). This in torn decreases a cross-section of the lumen (16) through which blood flows (12). In FIG. 1A, the narrowing caused by an atherosclerotic plaque (14) is of the order of 70% in the longitudinal view and, as such, would likely be treated with balloon angioplasty and stent placement. The cross-sectional view of FIG. 1A, shown at lower inset, illustrates the irregularly shaped lumen (16). As illustrated, plaques (14) commonly form with increased frequency at or very near to bifurcations of the artery (18).

In current practice, the cardiologist estimates the degree of narrowing as compared to the expected dimension of an open lumen. Based on this estimation, the cardiologist then, determines whether an angioplasty or coronary artery bypass graft (CABG—bypass surgery) is required to improve blood flow. Such stenotic lesions as plaques (14) are commonly treated using balloon angioplasty, as illustrated in FIG. 1B. A balloon catheter (22) includes a dilation balloon (20) attached to a guidewire (24) to navigate through the artery. The dilation balloon (20) is inserted at the area of the lesion (14) and inflated to increase the luminal size (16). Following dilation, stents which may be deployed by the use of the balloon catheter (22) are commonly placed at the site of the lesion to prevent stenosis at a later time (restenosis).

As known in the field, accurate stent expansion is critical to the success of an angiography procedure. For example, the over-expansion of a stent can cause rupture of the blood vessel. Conversely, under-expansion of a stent causes a region where blood flow is restricted and/or leaves gaps between a stent structure and the lumen wall, either condition of which can lead to thrombosis.

Accordingly, there is a need for a device, system, and method which provide for accurate measurement of the dimensions of a luminal space of an artery thereby allowing for the determination of the size to which a stent should be expanded. There is also a need for a method of using linear dimensions obtained by probing distances of an artery wall, from a device or system to determine a maximum diameter to which a stent, may be expanded, for example, determining such from a cross-sectional area.

### SUMMARY

The disclosure relates to a system and method for optically determining the shape and size of a lumen of a blood vessel, and of, for example, a balloon stent as it is inflated. The size and shape determination of the lumen of the blood vessel allows for accurate and safe deployment of the stent within an artery.

Thus, an embodiment of the disclosure is a stent delivery system. The system includes a catheter having a distal portion and a proximal portion; a guidewire removably received within the catheter: a plurality of optical emitting fibers, an expandable balloon disposed on the distal portion of the catheter and attached to or in communication or continuous with the catheter; and a stent. The plurality of optical emitting fibers for measuring a surrounding area in a lumen may be located on the guidewire. The guidewire may include a flexible tip at a distal portion of the guidewire. The stent may be disposed over an expanded portion of the balloon to a position within the measured surrounding area in the lumen. Alternatively the stent may be of the self-expanding variety in which a stent may be compressed by a sheath or other structure. When the sheath or other structure is retracted, for example, the compressed stent may expand to a predetermined diameter either with or without subsequent balloon dilation. In an alternative to this embodiment the stent delivery system may include a stent and a sheath, without an expandable balloon. In this instance, the size (diameter and length) of the self-expanding stent may be selected such that when the sheath or other structure is retracted the compressed stent may expand to a predetermined diameter which causes the stent to be fully or partially apposed against a lumen wall, depending upon the desired outcome,

The optical emitting fibers direct transmitted optical radiation to surrounding areas in the lumen and collect the optical radiation back from the surrounding, areas of the lumen. The optical radiation may be low coherence light, or light of any wavelength suitable to the various embodiments of the disclosure. Further, the plurality of optical emitting fibers may be single-mode or multi-mode fibers, and may be dispersed about a circumference of the guidewire. The optical emitting fibers may also include a central structure, which may be solid or hollow to allow delivery of fluid or gas to the balloon.

The system may further include a detector and processor. The detector may receive optical radiation back from the surrounding area of the lumen which is transmitted through the plurality of optical emitting libers. The processor may be in communication with the detector. The processor may control delivery of expansion gas or fluid to the balloon for expansion based on processing of the optical radiation signals provided by the plurality of optical emitting fibers. The balloon may be expanded or contracted by fluid or gas delivered through the catheter. The fluid may be any suitable optically transparent fluid, such as, for example saline, and may optionally contain a drug or other therapeutic substance.

The guidewire may preferably have a diameter of about 0.36 mm, while the catheter may preferably have a diameter of between about 1.0 mm and about 1.4 mm. The catheter may be single-walled or double-walled. The single-walled catheter may have an inner lumen which confines the guidewire to travel a defined path through the catheter. In the double-walled catheter, the guidewire may be confined to travel a path along the inner most lumen, while expansion fluid may flow through the outer lumen, which is defined by the first and second walls of the catheter. The double-walled, catheter may have openings at the distal end which allow delivery of gas or fluid to the balloon. The catheter may be designed such that the guidewire may be directed to transit externally to the proximal portion of the catheter and internally to the distal portion of the catheter.

Another embodiment of the disclosure is directed to a stent delivery system. The stent delivery system includes a catheter having a proximal portion and a distal portion; a guidewire removably received within the catheter: a plurality of optical emitting fibers; a balloon disposed on the distal portion of the catheter; and a stent. The plurality of optical emitting fibers for measuring a surrounding area in a lumen may be located on the catheter. These optical emitting fibers may be embedded within an outer lumen of the catheter, which may be single or double walled. Alternatively, the optical emitting fibers may be attached to the outer lumen of the catheter. Conversely the optical emitting fibers may be located on the guidewire or a combination of the guidewire and catheter. The stent may be deployed over the balloon to a position within the measured surrounding area in the lumen. Alternatively the stent may be of the self-expanding variety in which a stent is compressed by a sheath or other structure. When the sheath or other structure is retracted, the compressed stent may expand to a predetermined diameter either with or without subsequent balloon dilation. In an alternative to this embodiment, the stent delivery system may include a stent and a sheath, without an expandable balloon. In this instance, the size (diameter and length) of the self-expanding stent may be selected such that when the sheath or other structure is retracted the compressed stent may expand to a predetermined diameter which causes the stent to be fully or partially apposed against a lumen wall, depending upon the desired outcome.

The system may further include a detector and a processor. The detector may receive optical radiation hack from the surrounding area of the lumen which is transmitted through the plurality of optical emitting fibers. The processor may be in communication with the detector. The processor may control delivery of expansion gas or fluid to the balloon based on processing of the optical radiation signals provided by the plurality of optical emitting fibers.

The system may further have seals at ends of the balloon which ride over the guidewire and prevent leakage of expansion gas or fluid into the lumen. The catheter may be single-waited or double-walled. Expansion fluid may be pushed through the lumen of the single-walled catheter to expand the balloon, or may be pushed though the outer lumen of a double-wailed catheter to inflate the balloon. Alternatively, a wall of the catheter may include openings that allow delivery of gas or fluid to the balloon. The balloon may thus be expanded or contracted by fluid or gas pushed through the catheter, which may then exit through the openings to the balloon.

The guidewire may preferably have a diameter of about 0.36 mm, while the catheter may preferably have a diameter of between about 1.0 mm and about 1.4 mm. The catheter may be single-walled or double-walled. The catheter may be designed such that the guidewire may be directed to transit externally to the proximal portion of the catheter and internally to the distal portion of the catheter.

The optical emitting fibers may direct transmitted optical radiation to surrounding areas in the lumen and collect the optical radiation back from the surrounding areas of the lumen. The optical radiation may be low coherence light, or light of any wavelength suitable to the various embodiments of the disclosure. Further, the plurality of optical emitting fibers may be single-mode or multi-mode fibers, and may be dispersed about a circumference of the catheter.

Another embodiment of the disclosure is directed to a method for deploying a stent. The method includes introducing a stent delivery device having a plurality of optical emitting libers; measuring a surrounding area of the lumen; and actuating the stent delivery device. The stent delivery device may include a guidewire, a catheter, or a combination thereof, a stent, and an expandable balloon. The catheter may be located over the guidewire. The stem delivery device is actuated to deploy a stent to a portion within the measured surrounding area of the lumen. The method may further include transmitting and receiving optical radiation signals from the stent delivery device to determine a position of the stent in the target lumen either prior to the actuating step, after the actuating step or both.

The plurality of optical emitting fibers may located on the guidewire, the catheter or a combination thereof. The plurality of optical emitting fibers may be dispersed about a circumference of the structure. The optical emitting fibers may direct transmitted optical radiation to the surrounding area in the lumen and collect optical radiation back from the surrounding area of the lumen.

Actuating the stent delivery device to deploy the stent may include delivering gas or fluid to the stent delivery device. Further, introducing the catheter over the structure may occur after the guidewire is in the target lumen, or before the guidewire is introduced within the target lumen, such that the catheter and guidewire are positioned in the vessel at the same time. The stent delivery device may include a stent which may be of the self-expanding variety and a sheath or other structure which compresses the stent, and optionally an expandable balloon. When the sheath or other structure is retracted the compressed stent may expand to a predetermined diameter either with or without subsequent balloon dilation.

In yet another embodiment of the disclosure, a method of deploying a stent within a lumen is described. The method includes providing a stent delivery system into a body cavity or vessel lumen. The stent delivery system includes a catheter having a distal portion and a proximal portion; a guidewire removably received within the catheter, the guidewire having a plurality of optical emitting fibers for measuring a surrounding area in the lumen; a balloon disposed on the distal portion of the catheter; and a stent disposed over the balloon. The method may further include passing the catheter along the guidewire to place the stent at a desired position within the measured surrounding area in the lumen and deploying the stent within the measured surrounding area in the lumen.

Deploying the stent may further include the steps of calculating a set of optical pathlengths from returned optical radiation signals, the returned optical radiation signals received from the plurality optical emitting fibers and a lumen wall; determining a diameter of the lumen: and expanding the stent to at least a portion of the determined diameter of the lumen. Expanding the stent may be by delivery of expansion fluid to the balloon, such that the balloon may be expanded or contracted to the determined diameter. The optical system, which may be part of the guidewire or the catheter, may monitor balloon dilation in real-time to ensure that the stent has been fully expanded. Alternatively, a self-expanding stent of a predetermined diameter may be deployed within the lumen, where the size of the stent may be determined based on the measured diameter of the lumen. In this embodiment, the stent delivery system may include a stent and a sheath, and optionally an expandable balloon. In this instance, the size (diameter and length) of the self-expanding stent may be selected such that when the sheath or other structure is retracted the compressed stent may expand to a predetermined diameter which causes the stent to be fully or partially apposed against a lumen wall, depending upon the desired outcome. If an expandable balloon is part of the stent delivery system, the balloon may be used to further expand the stent if needed.

Another embodiment of the disclosure is directed to a method of deploying a stent within a body cavity or vessel lumen. A stent delivery system is placed within the body cavity or vessel lumen. The stent delivery system includes a catheter having a proximal portion and a distal portion, wherein the catheter includes a plurality of optical emitting fibers for measuring a surrounding area in the lumen; a guidewire removably received within the catheter; a balloon disposed at the distal portion of the catheter; a stent disposed over the balloon, and optionally a sheath. Alternatively, the plurality of optical emitting fibers may be located on the guidewire or a combination of the guidewire and the catheter. The method may further include passing the catheter along the guidewire to place the stent at a position within the measured surrounding area in the lumen and deploying the stent within the measured surrounding area in the lumen.

In all embodiments described and shown, the catheter may be disposed over the guidewire at a time before, concurrent with, or after the guidewire has been placed within the body cavity or vessel lumen. If the optical system is contained within a guidewire, as is described for several embodiments of the disclosure, placement of the guidewire within the body cavity or vessel lumen prior to deployment of the catheter and stent delivery system may allow for improved characterization of the surrounding tissue. If the optical system is contained within a catheter, as is described for several, embodiments of the disclosure, placement of the guidewire and catheter within the body cavity or vessel lumen prior to deployment of the stent delivery system may allow for improved characterization of the surrounding tissue. Once a lesion has been located which may require treatment, the stent delivery system, which may include a balloon expandable stent, a self-expanding stent, or equivalent known in the art may be deployed over the guidewire or catheter.

In the case of a balloon expandable stent, the optical emitting fibers direct transmitted optical radiation to the surrounding area in the lumen and collect optical radiation back from the surrounding area of the lumen, allowing for determination of the vessel lumen size. Further, the measurement of the vessel lumen, diameter and lesion size may allow for an accurate selection of the appropriate size (diameter and length) stent. This selected stent may then be deployed using a stent delivery system to the correct location within the vessel lumen. The balloon may be expanded or contracted by fluid or gas delivered through the catheter to cause expansion of the stent to the measured vessel lumen size. The optical system, which may be part of the guidewire or the catheter, may monitor balloon dilation in real-time to ensure that the stent has been folly expanded.

In the case of self-expanding stents, measurement of the vessel lumen diameter and lesion size may allow for an accurate selection of the appropriate size (diameter and length) stent. This selected stent may then be deployed using a stent delivery system, which may include the stent, and a sheath, to the correct location within the vessel lumen. The optical system, which may be part of the guidewire or the catheter, may then allow for a determination, after stent deployment, of whether balloon dilation may be required to achieve the desired diameter/cross-sectional area of the stent, and to monitor balloon dilation, for example, in real-time to ensure that the stent has been fully expanded.

### BRIEF DESCRIPTION OF THE SEVERAL VIEWS OF THE DRAWINGS

The file of this patent contains at least one drawing executed in color. Copies of this patent with color drawings will be provided by the Patent and Trademark Office upon request and payment of necessary fee.

For a better understanding of the disclosure and to show how the same may be carried into effect reference will now be made to the accompanying drawings. It is stressed that the particulars shown are by way of example only and for purposes of illustrative discussion of the preferred embodiments of the present disclosure only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings;

FIG. 1A illustrates a longitudinal (top) and a cross-sectional (bottom) view of a stenosis in an artery near an arterial bifurcation.

FIG. 1B illustrates a longitudinal and a cross-sectional view of a dilation balloon showing compression of the stenotic lesion (top) and a cross-sectional view of the widened lumen after the balloon is deflated and withdrawn (bottom).

FIG. 2 is a schematic of a stent delivery system of the present disclosure, including a balloon catheter inserted over a guidewire that incorporates one or more optical probes.

FIG. 3 illustrates a longitudinal (top) and a cross-sectional (bottom) view of a partially expanded balloon stent of the present disclosure, disposed over an optical probe guidewire at a lesion site.

FIG. 4 illustrates alignment of an optical probe system of the present disclosure with a physical arterial structure it is sensing (bottom) and a corresponding LCI signal trace (top).

FIG. 5 is an LCI trace from an embodiment of the present disclosure in arterial tissue.

FIG. 6 illustrates progression of an LCI signal as a balloon advances from an unexpanded initial state (top) to a point of stent deployment at an artery wall (bottom).

FIG. 7 illustrates a cross-sectional view of a six-probe guidewire embodiment with a balloon and stent partially expanded.

FIG. 8 illustrates dimensions to which a stent of an embodiment of the present disclosure may be deployed.

FIG. 9A illustrates a catheter embodiment showing a balloon device deployed over a guidewire.

FIG. 9B is an expanded view of an optical emitter of a catheter based device without a guidewire for clarity.

FIG. 9C is a close up view of another embodiment, of a catheter device.

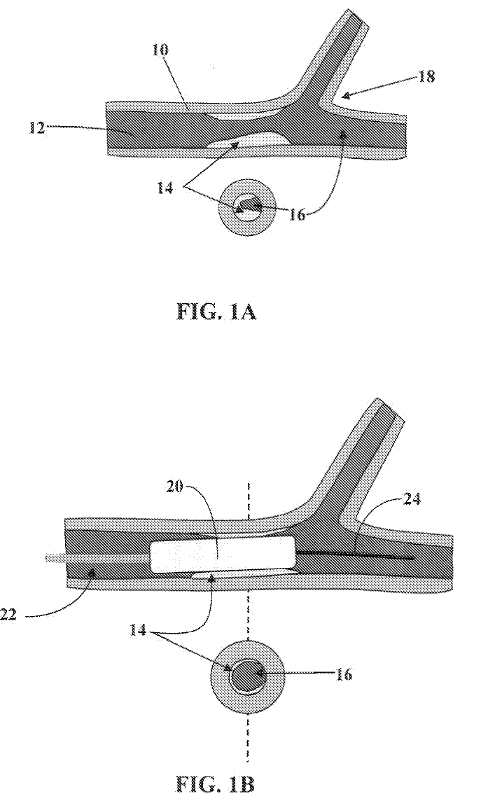

### DETAILED DESCRIPTION

Before the present devices, systems and methods are described, it is to be understood that this invention is not limited to the particular processes, devices, or methodologies described, as these may vary. It is also to be understood that the terminology used in the description is for the purpose of describing the particular versions or embodiments only, and is not intended to limit the scope of the present disclosure which will be limited only by the appended claims.

It must also be noted that as used herein and in the appended claims, the singular forms “a”, “an”, and “the” include plural reference unless, the context clearly dictates otherwise. Thus, for example, reference to an “artery” is a reference to one or more arteries and equivalents thereof known to those skilled in the art, and so forth. Unless defined otherwise, all technical and scientific terms used herein have the same meanings as commonly understood by one of ordinary skill in the art. Optional” or “optionally” means that the subsequently described structure, event or circumstance may or may not occur, and that the description includes instances where the event occurs and instances where it does not.

The term “plaque” may be taken to mean any localized abnormal patch on a body part or surface. In regard to arterial plaques, plaques may be fatty deposits on the inner lining of an arterial wall and are characteristic of atherosclerosis. The plaque may be an abnormal accumulation of inflammatory cells, lipids and a variable amount of connective tissue within the wails of arteries. In part, embodiments of this invention are directed to the detection and treatment of plaques.

Although any methods and materials similar or equivalent to those described herein can be used in the practice or testing of embodiments of the present disclosure, the preferred methods, devices, and materials are now described. Nothing herein is to be construed as an admission that the invention is not entitled to antedate such disclosure by virtue of prior invention.

The disclosure generally relates to a device, system and method for optically determining a shape and size of a lumen of a blood vessel. The disclosure also generally relates to a system and method for optically determining a shape and size of a balloon stent as it is inflated within that lumen. The determination of size, preferably a precise size, of a blood vessel allows for accurate and safe deployment of for example, a balloon catheter or stent, within an artery.

An embodiment of the disclosure is directed to a catheter for use in measurement of dimensions of an arterial lumen and accurate deployment of a stent into such region. The catheter device generally includes a plurality of optical emitting fibers which may be contained within a guidewire structure or within the catheter structure. The guidewire may be about 0.014 inches in diameter which is representative of current guidewire sizes used for coronary applications as understood by one skilled in the art. Alternatively, the size of the guidewire may vary depending on the desired application. For example, a guidewire and optical emitting fibers may be contained within a balloon catheter. The balloon catheter may be a hollow tube that is introduced over the guidewire. The balloon catheter may be approximately 1 mm in diameter for coronary applications. An appropriately sized stent may then be disposed over the balloon catheter. The balloon may be expanded or contracted by fluid or gas delivered through the catheter. Alternatively the stent may be of the self-expanding variety in which a stent is compressed by a sheath or other structure. When the sheath or other structure is retracted the compressed stent may expand to a predetermined diameter either with or without subsequent balloon dilation. All reference to a “stent” in the present disclosure may be taken to include both deformable non self-expanding stents and self-expanding stents,

Another embodiment of the disclosure is directed to a method for determining a size of a vessel lumen by use of optical radiation. The method includes utilizing optical radiations of a short coherence length (approximately 20 μm, or preferably shorter for semiconductor light-emitting diode sources). This allows the determination of linear dimensions of a lumen with a precision of about the coherence length. While the preferred optical embodiment is based on low-coherence interferometry (LCI), other techniques operating in tins wavelength range also may be used. The LCI backseattered signal, which allows the size determination of an artery, may also be used to determine the linear distance of the optical emitting fiber to a stent disposed on a balloon, as well a linear distance to the lumen wall. These linear dimensions, which are obtained by analysis of backscattered light received by the optical emitting fibers allows for the determination of, for example, a cross-sectional area and, from that area, the diameter to which a stent should be expanded.

A further embodiment of the disclosure is directed to a method of using received backscattered fight from optical emitting fibers and calculating dimensions from such data to determine the size of a stent expansion in real time as well as the size of the lumen. By use of feedback or other signal processing in real-time, stent expansion may be stopped during a process when a desired expansion size is achieved without exceeding a maximum diameter of the lumen. For example, the stent expansion may be controlled manually by a physician or alternatively may be controlled by an automated software system. Additionally, the software system may include a fail safe mechanism, whereby expansion of a stent can not exceed a maximum size, the maximum size being the measured diameter of the lumen of the artery.

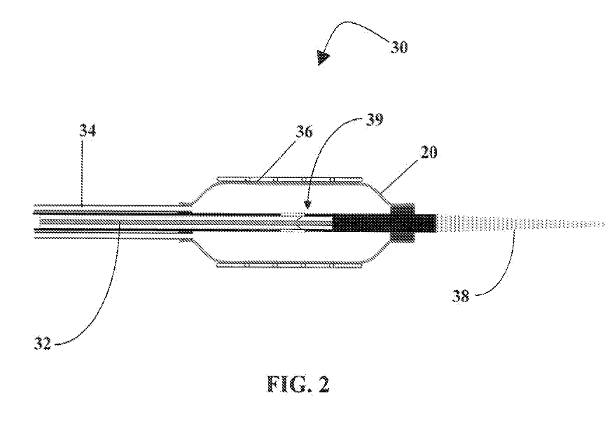

Turning now to the figures, FIG. 2 illustrates a device (30) of an embodiment of the disclosure. The device (30) may include a guidewire (32), a catheter (34), and a stent (36), Guidewire (32) may be of any type and size. For example, the size of guidewire (32) may be one known and used in the industry such as about 0.014 inches in diameter. The catheter (34) rides over guidewire (32) as is practiced and understood by one skilled in the art. A balloon (20) is attached to or in communication or continuous with the catheter (34) and rides over the guidewire (32). The balloon (20) may be used to expand and place the stent (36) into the desired area. The balloon may be inflatable through the catheter, with a common inflation fluid being saline solution or by any other manner as known and understood by one skilled in the art. The guide wire (32) terminates at a distal end in a flexible tip (38) which facilitates navigation of the guide wire (32) to the particular artery being examined and/or treated. The guide wire (32) includes one or more optical probes (39), with each optical probe containing one or more optical emitting fibers. In a preferred embodiment, the guide wire may include six optical probes as disclosed in corresponding U.S. patent application Ser. No. 11/191,097 entitled Device for Tissue Characterisation, which is incorporated by reference in its entirety herein.

Any suitable balloon expandable stent, self-expanding stent, or equivalent known in the art may be used in the stent delivery systems in accordance with the present disclosure. Also, the above description is provided merely to illustrate one example of an inflation-type stent delivery system suitable for use in embodiments of the present disclosure, and other now-known or later developed inflation-type stent delivery systems or self-expanding systems may also be used to form a stent delivery system in accordance with the present disclosure.

Balloons used in the stent delivery systems in accordance with the present disclosure, are well known and, thus, although described and shown with reference to a preferred embodiment, the general features (e.g. size, shape, materials) of the balloon may be in accordance with conventional balloons. In a preferred embodiment, the balloon may be made of an optically transparent, flexible medical-grade silicone rubber which is capable of being inflated to any volume and length as required by embodiments of the present disclosure. Alternatively, the balloon may be made of other materials, such as polyethylene terepthalate (PET), polytetrafluoroethylene (PTFE) or polyethylene; most preferably a material that is optically transparent to the optical radiation, biocompatible, and distendable. Modern percutaneous transluminal-coronary angioplasty (PTGA) balloons are also made of Pebax® or any other nylon tubing suitable for such applications.

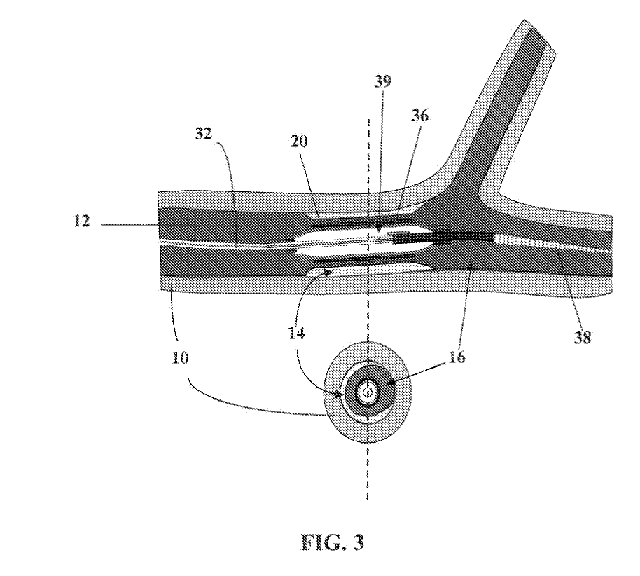

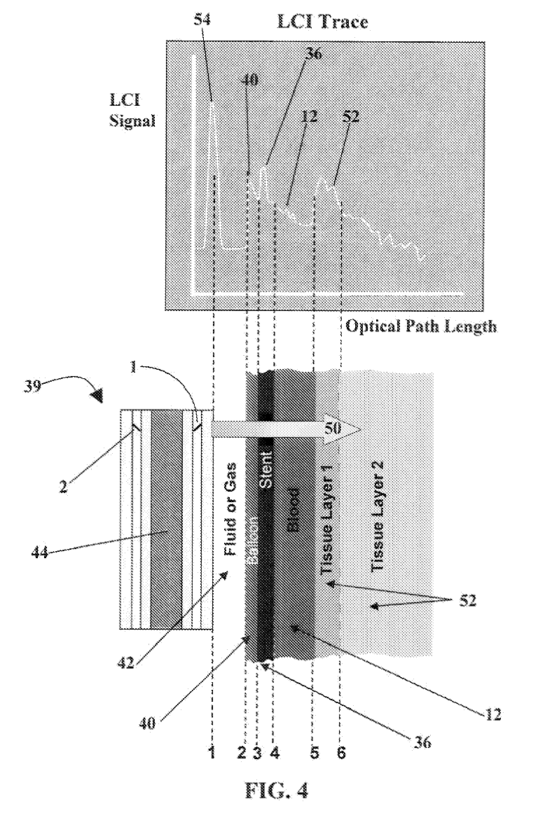

FIG. 3 illustrates a stent (36) and guidewire (32) containing an optical probe (39) of an embodiment positioned at a site of a lesion (14). The balloon (20) is partially inflated in this view, in this embodiment, the guidewire (32) includes optical probe (39) which contains multiple optical emitting fibers, each of which terminates in an optical head that deflects and, possibly shapes the emitted optical radiation pattern, in a preferred embodiment, six optical emitting fibers may be used to generate six optical beams which may be directed at or along the circumference of the lumen (16). The center of each optical beam pattern on the inner wall of the lumen may be equally spaced from the adjacent, beam patterns. That is, for six beams, each, beam is about 60° from each other. Alternatively, the multiple beams may be closely spaced together, or may be spaced further apart, depending on the desired, area to be examined. Therefore, the beams may be spaced evenly about a lumen, i.e. 60° apart or may be placed unevenly apart. For example, all six probes may be located within a 90° area.

As used herein, “optical emitting fibers” refers to optical fibers that are typically made of glass or a material having a higher dielectric constant than the surrounding medium. The dielectric constant can be constant across the diameter of the fiber or it can follow a particular profile across the diameter of the fiber. In addition, “optical emitting fibers” also includes hollow, air-filled tubes with reflecting inner walls, and hollow tubes surrounded by a honeycomb structure of other hollow tubes.

Whether wave propagation in the fiber is single-mode or multi-mode is immaterial to the practice of the various embodiments of the disclosure. Hence, the term “optical emitting fibers” is also intended to include single-mode or multi-mode fibers. Single mode fibers may be preferable for maximizing longitudinal resolution. However, multimode fibers may be smaller in size and thus maximize radial resolution and device flexibility. Average sizes for single mode fibers may be on the order of about 100 μm diameter, while an average catheter diameter may be about 1 to 3 mm. Thus, a maximum of about 30 to 100 single mode fibers may be used. In a preferred embodiment, 1-12 optical fibers may be utilized, more preferably 1-6 optical fibers.

In addition, the polarization of the wave propagating on the fiber is immaterial to the practice of various embodiments of the disclosure. Hence, the term “optical emitting fibers” includes within its scope waveguides that display birefringence or other properties that are associated with polarization of waves propagating in the waveguide. Embodiments of the disclosure are not restricted to infrared radiation but may be equally amenable to electromagnetic radiation having wavelengths outside the infrared range. In particular, electromagnetic radiation at optical frequencies may be used. Although this detailed description teaches one particular embodiment in which measurements are made in the infrared range, the scope of the invention is not limited to infrared frequencies.

With continued reference to FIG. 3, the optical emitting fibers within the optical probe (39) receive light scattered back from tissue on the inner surface of the lumen (16) and/or within the artery wall (10). In the case where low coherence interferometry (LCI) is used, the optical emitting fibers may receive scattered light from the blood (12), the artery wall (10), and tissue within the artery itself, which may include plaques (14) or other structures, in addition to the structural elements of the balloon (20) and stent (36) as shown in FIG. 3.

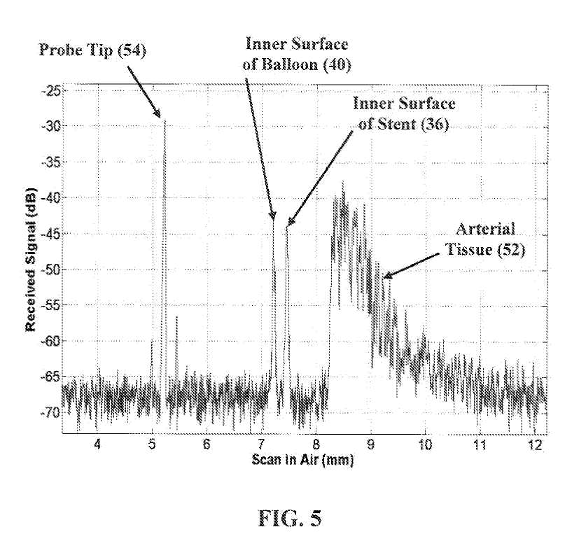

The backscattered light received at the optical emitting fiber is illustrated in graphical form in FIG. 4, where guidewire (32) is located in an artery (oriented 90° from the view in FIG. 3) and is emitting light (50) into blood (12) and various layers of arterial wall material (52) (for illustration purpose, two tissue types are represented). FIG. 4 illustrates an optical emitting fiber (1) and an opposed optical emitting fiber (2) in optical probe (39), in addition to central member (44) of the distal end of the guidewire. Note, while only two optical emitting fibers are shown, it is merely for illustrative purposes and multiple optical emitting fibers may be included in the optical probe (39). Central member (44) may be solid or alternatively hollow to allow for delivery of fluid, gas drug, or the like. FIG. 4 shows an. LCI trace aligned with the physical features of the optical probe which has a balloon and stent thereon, and the artery. These alignments are numbered 1 through 6 at the bottom of the figure to correspond to the features of the LCI trace depicted above. The LCI response is characterized by the following signal components:
(1) Reflection of light from the optical emitting fiber edge along optical path 50, at the interface between the glass of the optical emitting fiber and clear fluid or gas (42), which is used to flush or inflate the balloon. This feature is labeled as 54 in the LCI trace.
(2) Backscattered light from the edge of the optical emitting fiber along optical path 50 at the interlace between the clear fluid (42) and the inner balloon wall (40). Light scattering from the balloon material contributes signal until the outer wall of the balloon is reached. This feature is labeled as 40 in the LCI trace,
(3) Backscattered light from the edge of the optical emitting fiber along optical path 50, from the inner wall of the stent (36) which is typically metallic and highly reflective. The backscattered signal between lines 3 and 4 is from blood (12) that fills the spaces between the struts of the expanding stent. As such, backscattered light from the inner wall of the metallic stent (36) will decrease as the stent is expanded, and the backscattered light from the blood (12) which fills the spaces between the struts of the stent will increase as the stent is expanded. This feature is labeled as 36 in the LCI trace.
(4) Backscattered light from the edge of the optical emitting fiber along optical path 50, with the outer wall of the stent (36). Note, no light penetrates the stent itself, rather the LCI signal is scattering from the blood (12) that fills the spaces between the struts of the expanding stent. The thickness of the struts is known from the design specifications of the thickness of the stent.
(5) Backscattered light from the edge of the optical emitting fiber along optical path 50, aligned with the blood (12) to lumen (16) interface, from which, the first (leftmost, on the curve) LCI signals from lesions (14) or arterial tissue (52) will emanate.
(6) Backscattered light from the probe tip along path 50, aligned with the blood (12) to lumen (16) interface, between two types of tissue (52), e.g., a fibrous cap and a necrotic core that comprise the arterial wall section being probed.

An example LCI trace from arterial tissue is shown in FIG. 5. The LCI signals from the optical fiber probe tip (54), the inner surface of a balloon (40) the inner surface of a stent (36), and arterial tissue (52) are shown. In this example, the balloon is approximately 2 mm from the probe tip, measured as the distance between the probe tip LCI signal (54) and the signal from the inner surface of the balloon (40). The balloon is in contact with the inner surface of the stent, and its diameter may be determined by the distance between the signals from the inner surface of the balloon (40) and the inner surface of the stent (36). Tissue from the artery is about 0.8 mm from the inner surface of the stent, as is seen by the distance between the signals from the inner surface of the stent (36) and the arterial tissue (52). The LCI signal is observed to penetrate into the arterial tissue for about 2 mm. This data is taken through air, therefore all signals from blood are absent (as would be observed at the 4-5 interface in FIG. 4),

An alternate embodiment of the disclosure may have the probe located within the guidewire with a balloon riding over the guidewire, but with no stent on the balloon. In this embodiment, the interfaces at positions 3 and 4 of FIG. 4, corresponding to the stent, would not be present. The LCI signal, in this region would derive primarily only from the scattering from blood (12). In such a case, the refractive index of blood, nₘ(4-5) (where “m” is the number of optical emitting fibers located in the optical probe and preferably is between 1-100), would be used to determine distances between the balloon wall and the arterial tissue. Here, the designation nₘ refers to the refractive index of fluid in the sensing region of the m^{th} fiber probe and “4-5” refers to the region between interfaces 4 and 5 (as discussed herein below). Note that such an embodiment implies a subsequent deployment of a stent (36) on a balloon catheter over the guidewire. This would allow for a more accurate determination of lesions within an artery and stent deployment,

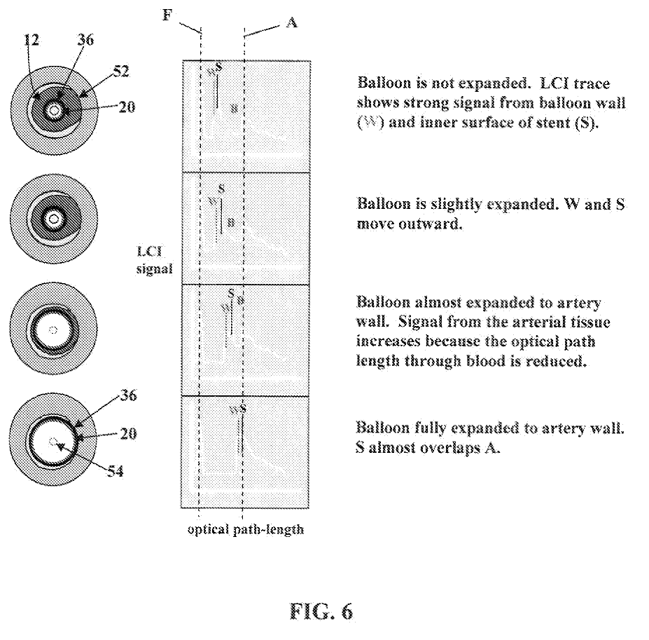

The embodiment disclosed and illustrated in FIG. 4 may generate a progression of signals as the balloon expands within the artery as is illustrated in FIG. 6. The positions and intensities of the features in the LCI signal shown in FIG. 4 change as the balloon is expanded. The signal features are labeled in FIG. 6 as follows: F=edge of optical emitting fiber (54): W=inner wall of the balloon (20); S=inner surface of stent (36); B=blood (12); A=arterial tissue (52).

The distance between the edge of optical emitting fiber (F) and arterial tissue (A) may be constant for any given position of the optical probe along the length of the vessel being examined. The distance between the inner balloon wall (W) and the inner surface of the stent (S) decreases slightly as the balloon expands and thins in the expansion process. The signal from the arterial tissue, which may be made of several layers or components (52) (only one layer of arterial tissue is shown), increases as the balloon (20) expands and less blood (12) is transversed by photons emitted from the optical emitting fiber and detected by the optical emitting fiber. This signal increases due to a reduction in the losses from backscattering at interfaces or scattering from within the blood at the pathlength being interrogated. The distances to the outer wall of the stent (interfaced 4 in FIG. 4) and the blood-tissue interface (interface 5 in FIG. 4) from the edge of the optical emitting fiber may be determined from the LCI trace.

The physical distances from any interface j to any other interface k (where “j” and “k” are any of the interfaces (1-6) illustrated in FIG. 4) along the light-path of the m^{th} optical emitting fiber of the optical probe are designated as dₘ(j−k). For example, the physical distance from the edge of the first optical emitting fiber to the blood/artery wall interface is d(1-5), as designated by FIG. 4. The optical pathlength is similarly designated as lₘ(j−k); which in the specific example cited above would be |(1-5). In general, for any segment of the light path, the optical pathlength is equal to the physical distance multiplied by the index-of-refraction of the material between those interfaces, nₘ(j−k). Such indices may be renamed in the following text for simplicity and clarity. For example, nₘ(j−k) where j=4 and k=5 would be named n_{blood}.

The position of the outer wall of the stent may be accurately measured by adding the thickness of the stent (known from design specifications) to the distance 1-3 or, alternatively, adding the thicknesses of the balloon (which may vary depending on the degree of” inflation and the balloon design) and the stent to the distance 1-2. The signals from 1, 2, and 3 are dominated by reflectance rather than scattering so they may be measured to within the accuracy of the measurement system. For LCI, this is the coherence length of the illumination source, typically in the 10-30 μm range (but can be as small as ˜1 μm-using an extremely broadband light source). If scattering from the region 1-2 is used to determine distance, the dimension determined from the LCI trace may be multiplied by n_{fluid} (the index-of-refraction of the expansion fluid at the wavelength used) to define an accurate distance.

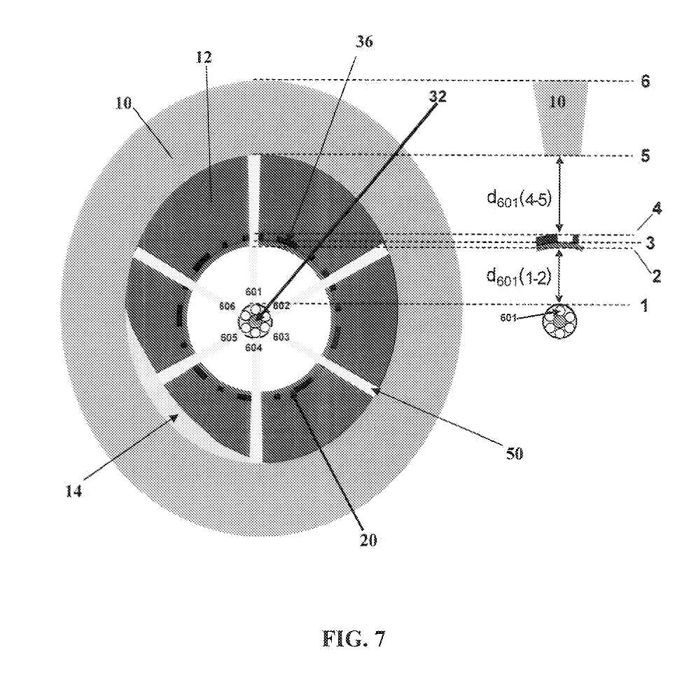

FIG. 7 illustrates a cross-sectional view of a guidewire embodiment of the disclosure with the balloon (20) partially expanded. The numbers 1, 2, 3, 4, 5 and 6 refer to the interfaces previously identified in FIG. 4. The optical emitting fibers are labeled 601 through 606 in this 6-probe embodiment. The distance from, the edge of m^{th} optical emitting fiber to the blood/tissue interface is designated as dₘ(1-5). In the LCI trace in FIG. 4, the first (rightmost) signals from interface 5 are due to direct backscattering and are equal to the optical pathlength, lₘ(3-5). The distance dₘ(3-5) may be obtained by multiplying the optical pathlength by the index-of-refraction of blood at the wavelength used (n_{blood}).

Note that the embodiment of the stent (36) illustrated in FIG. 7 is not a continuous cylinder. It is a mesh, the details of which may depend on the specific design specifications and may vary in material or design as understood by one skilled in the art. Before the balloon (20) is expanded, the optical light path (50) from the optical emitting is substantially or in some instance completely occluded by the highly reflective surface of the compressed stent. As the balloon expands, however, some light will pass through the struts of the stent to interrogate the artery wall (10). The signal from interlace 4 will diminish as the stent expands, both due to the increased distance from the edge of the optical emitting fibers to the stent (the backscattered signal, S, is proportional to d⁻²) and also due to the decreasing ratio of light reflected by the stent to that which passes through it.

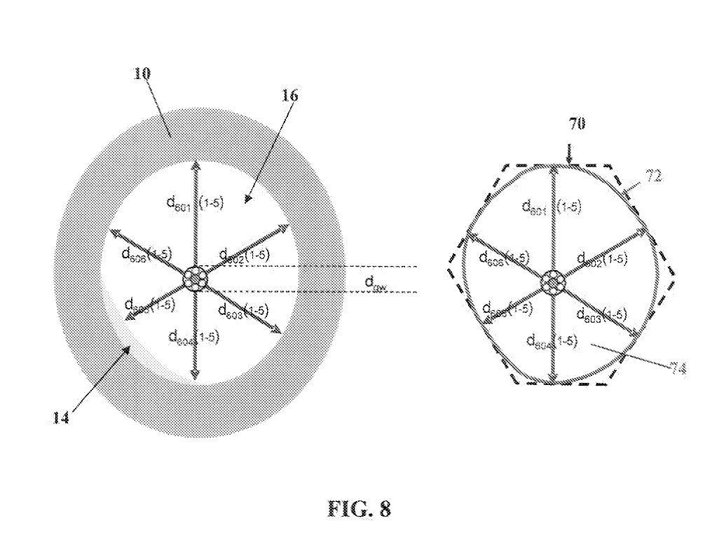

FIG. 8 illustrates the m radial distances, dₘ(1-5), for all of the fibers of a six optical emitting fibers probe embodiment of the disclosure. Also illustrated in FIG. 8 is the diameter of the guidewire, d_{gw}. These dimensions allow for a determination of a polygon (70; hexagon in the case of 6 optical emitting fibers as shown) from which a smoothed periphery (72) may be approximated by various mathematical techniques. An example of a mathematical approach is to compute the lumen area Aₛ (74) and then its diameter D. The total area may be computed, using the sine and cosine laws and the observed (measured) distances dₘ. For the example shown in FIG. 8 the angle between each of the optical emitting fibers is 60°, thus:

D=d_{gw}+Σdₘ/3 (1)

The formula above may be generalized to any number of optical emitting libers, m. The area Aₛ (74) enclosed by the smoothed periphery (72) may be calculated by many methods as used in the art. A circle of equivalent area would have a diameter, D, such that:

$$D = {[ \frac{4 {A}_{S}}{π} ]}^{\frac{1}{2}} \qquad ( 2 )$$

### This is the diameter to which the stent should be expanded.

In yet another embodiment of the disclosure, to automate the operation, the positions of stent (36) and arterial tissue wall (52) may be derived by processing LCI signals of the types shown in FIGS. 4, 5 and 6. These positions may be fed back to a mechanism that controls the introduction and withdrawal of expansion fluid through a catheter to the balloon. It is important, however, to note that feedback may not be necessary. All information needed to stop or prevent stent expansion is already in the data collected, which also contains information about the location of the stent. From this data, one may use the same approach described above to compute the effective diameter Dₛ of the stent and compute the value, of D−Dₛ in real time. The instrument may be programmed using, for example, a software program may be programmed to stop the balloon expansion when D−Dₛ is close to zero.

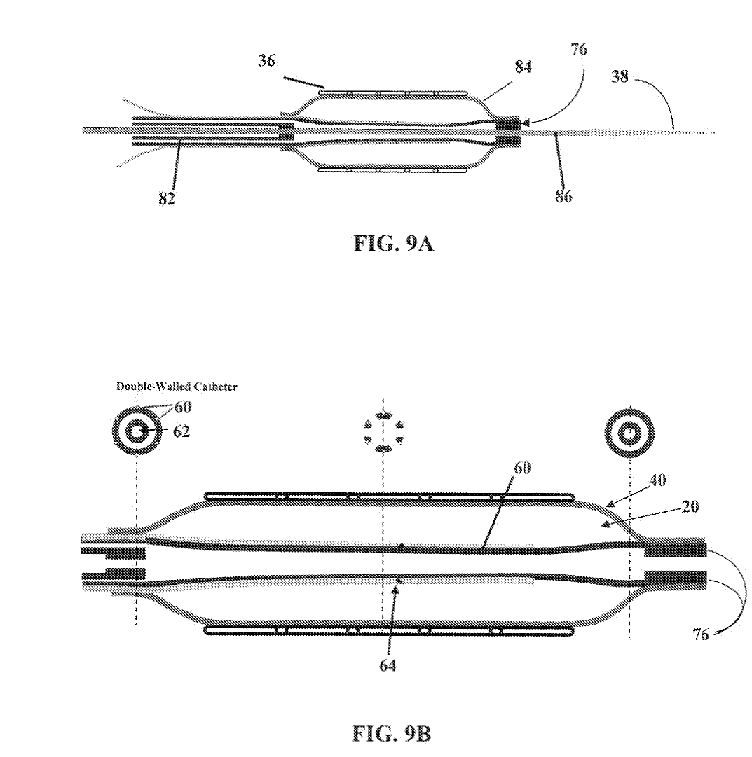

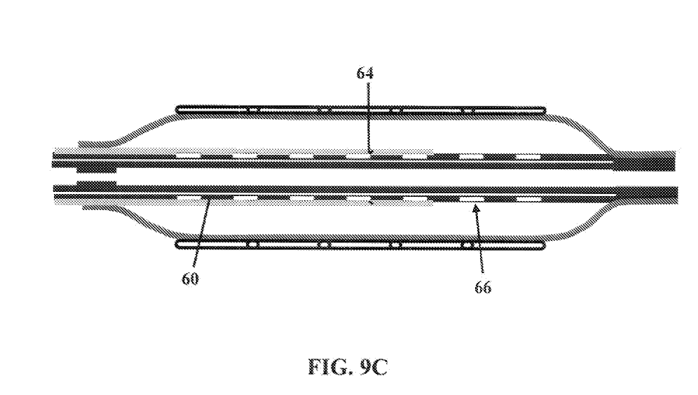

In another embodiment, the multiple optical emitting fibers disclosed in FIGS. 4, 7 and 8 may also be configured to be part of a catheter-based device that is deployed over a standard non-optical guidewire (24). Embodiments of this type of device are shown in FIG. 9.

In the embodiment of the device illustrated in FIGS. 9A, 9B and 9C, expansion fluid may be introduced between the inner and outer walls of a double-walled catheter (82). In these embodiments, the optical emitting fibers (60) may be embedded or located within the outer wall of the double wailed catheter. Each optical emitting fiber has a beam shape element (64), which may be a mirror, diffractive device, or include refractive or other reflective elements to shape the optical beam. In the embodiment of FIG. 9A, seals (76) at either end of the balloon (84) may ride over guidewire (86) and prevent significant leakage of the expansion fluid into the lumen of the vessel. The inner tube of this double walled catheter embodiment may contain the guidewire, while the outer tube may allow for delivery of the inflation fluid or gas. The balloon seals to the outer lumen proximally and the inner guidewire lumen distally. Inflation medium flows through the space between the inner and outer lumens and into the balloon.

An alternate embodiment is shown in FIG. 9B. Cross-sections of the catheter structure with the optical emitting fibers are shown in the three insets above the main FIG. 9B. The central portion, of the catheter (82) has holes or slots to pass the expansion fluid to the balloon, while the leftmost and middle portions also include the optical emitting fibers (60).

An alternate embodiment of this catheter device is shown in FIG. 9C. Here the catheter device is a double walled catheter in which the catheter may extend through the length of the balloon. In this embodiment, the outer wall of the catheter may be perforated with holes or slots (66) to allow the expansion fluid to fill the balloon. This embodiment may also contain optical emitting fibers (60) and beam shaping element (64). In all embodiments shown in FIG. 9, the optical emitting fibers may be placed such that the optical path is through the region of the balloon (82 of FIG. 9A, 40 of FIG. 9B) on which the stent (36) is disposed.

It should be noted that the beam shaping element in either guidewire or catheter embodiments may include optical elements not shown explicitly. This may include, for example, refractive or diffractive (e.g., holographic) elements either to shape the exiting beam or reflective or diffractive (e.g., holographic) elements to redirect the light towards the vessel wall.

In all embodiments described and shown, the catheter may be deployed over the guidewire at a time before, concurrent with, or after the guidewire has been placed within the body cavity or vessel lumen. If deployed after the guidewire, once a lesion has been located which may require treatment the catheter and stent delivery system, which may include a balloon expandable stent, a self-expanding stent, or an equivalent known in the art may be deployed over the guidewire. If deployed concurrent with the guidewire, once a lesion has been located which may require treatment, the stent delivery system, which may include a balloon expandable stent, a self-expanding stent, or an equivalent known in the art may be deployed over the catheter. Alternatively, the guidewire, catheter and stent delivery system may be placed within the vessel lumen simultaneously.

In all embodiments described and shown, the stent delivery system may include a catheter having a proximal portion and a distal portion; a guidewire removably received within the catheter; a stent; optionally a balloon disposed at the distal portion of the catheter; and optionally a sheath disposed over the stent. The stent may be a balloon expandable stent, a self-expanding stent, or an equivalent known in the art. In the case of a balloon expandable stent, optical emitting fibers, which may be part of the guidewire or the catheter, may direct transmitted optical radiation to the surrounding area in the lumen and collect optical radiation back from the surrounding area of the lumen, allowing for determination, of the vessel lumen size. Measurement of the vessel lumen diameter and lesion size may also allow for an accurate selection of the appropriate size (diameter and length) balloon expandable stent, although such selection may not be required. This selected stent may then be deployed using the stent delivery system to the correct location within the vessel lumen. The balloon may be expanded or contracted by fluid or gas delivered through the catheter to cause expansion of the stent to the measured vessel lumen size. The optical system may monitor balloon dilation in real-time to ensure that the stent has been fully expanded.

In the case of a self-expanding stent, measurement of the vessel lumen diameter and lesion size may allow for an accurate selection of the appropriate size (diameter and length) stent. This selected stent may then be deployed using a stent delivery system, which may include the stent, a sheath, and optionally a balloon, to the correct location within the vessel lumen. The optical system, which may be part of the guidewire or the catheter, may then allow for a interrogation, after stent deployment, of the deployed stent and the surrounding area. In an embodiment which includes a balloon, the optical system may allow for determination of whether balloon dilation may be required to achieve the desired diameter/cross-sectional area of the stent, and to monitor balloon dilation, for example, in real-time to ensure that the stent has been fully expanded.

The several embodiments of the present disclosure offer numerous advantages. The accurate placement of a stent using the systems and devices disclosed herein reduce the risk of stent over-expansion and artery rupture. Further, the accurate placement may reduce the risk of stent under-expansion and the incidence of late thrombosis. The systems and methods presented herein allow for accurate placement and deployment of a stent into a body cavity or vessel lumen based on determination of the size and shape of the lumen. As such, the stent placement may be controlled to allow for full deployment or partial deployment. The stent placement may also be controlled to allow for correct placement and deployment in irregularly shaped lumens, thus further reducing the risk of either over or under-expansion.

The systems and methods presented herein integrate diagnostic techniques in the use of low coherence interferometry or other imaging system to monitor the location of a lesion, and therapeutic techniques in the use of a balloon catheter system for the accurate placement and deployment of a stent at the location of a lesion. As such, the embodiments of the present disclosure eliminate the need for flushing solutions or other imaging enhancement methods that may be problematic to patient health. The expansion gas or fluid of the present system, is delivered to the balloon, thus providing a cleared imaging field without introducing solutions into the body cavity or vessel lumen that may dilute the blood or other body fluid, leading potentially to ischemia, electrolyte imbalance or congestive heart failure.

The application of the present systems and methods in the field of cardiovascular therapy is only one of the possible applications for the present invention. Minimally invasive surgery is applied in many fields of medical diagnosis and therapy, such as in other vascular, breast, urethral and renal, and abdominal procedures, for example, and the present invention may be applied in these fields.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub combination.

It will be appreciated by persons skilled in the art that the present invention is not limited to what has been particularly shown and described hereinabove. Rather the scope of the present invention, is defined by the appended claims and includes both combinations and sub combinations of the various features described hereinabove as well as variations and modifications thereof which would occur to persons skilled in the art upon reading the foregoing description.

What is claimed is:

1. A stent delivery system, comprising:
a catheter having a distal portion and a proximal portion;
a guidewire removably received within the catheter;
a plurality of optical emitting fibers for measuring a surrounding area in a lumen wherein the plurality of optical emitting fibers is located on the catheter, the guidewire, or a combination thereof;
an expandable balloon disposed on the distal portion of the catheter; and
a stent disposed over an expanded portion of the balloon to a position within the measured surrounding area in the lumen.

2. The system according to claim 1, wherein the plurality of optical emitting fibers direct transmitted optical radiation to the surrounding area in the lumen and collect optical radiation back from the surrounding area of the lumen.

3. The system according to claim 2, wherein the optical radiation is low coherence light.

4. The system according to claim 1, further comprising:
a detector, wherein the detector receives optical radiation back from the surrounding area of the lumen which is transmitted through the plurality of optical emitting fibers; and
a processor in communication with the detector, wherein the processor controls delivery of expansion gas or fluid to the balloon for expansion from information obtained by processing of the optical radiation signals provided by the plurality of optical emitting fibers.

5. The system according to claim 1, wherein the guidewire further comprises a flexible tip at a distal portion.

6. The system according to claim 1, wherein the balloon is expanded or contracted by fluid or gas delivered through the catheter.

7. The system according to claim 1, wherein the plurality of optical emitting fibers are dispersed about a circumference of the guidewire or of the catheter.

8. The system according to claim 1, wherein the plurality of optical emitting fibers includes a central structure.

9. The system according to claim 8, wherein the central structure is solid,

10. The system according to claim 8, wherein, the central structure is hollow to allow delivery of fluid or gas to the balloon.

11. The system according to claim 1, wherein the plurality of optical emitting libers are single-mode or multi-mode fibers.

12. The system according to claim 1, wherein the catheter is a double-walled catheter comprising openings on an inner wall at the distal portion of the catheter.

13. The system according to claim 12, wherein the openings allow delivery of gas or fluid to the balloon.

14. The system according to claim 1, further comprising seals at ends of the balloon which ride over the guidewire, wherein the seals prevent leakage of expansion gas or fluid into the lumen.

15. The system according to claim 1, wherein the balloon is continuous with the catheter.

16. The system according to claim 1, wherein the balloon is optically transparent.

17. A method for deploying a stent comprising:
introducing a stent delivery device into a lumen, wherein the stent delivery device comprises a plurality of optical emitting fibers;
measuring a surrounding area of the lumen; and
actuating the stent delivery device to deploy a stent to a portion within the measured surrounding area of the lumen.

18. The method according to claim 17, wherein the stent delivery device comprises a guidewire, a catheter, or a combination thereof, a stent and an expandable balloon.

19. The method according to claim 18, wherein the plurality of optically emitting fibers is located on the guidewire, the catheter, or the combination thereof.

20. The method according to claim 19, further comprising the step of transmitting and receiving optical radiation signals from the stent delivery device to determine a position of the stent in the lumen either prior to the actuating step, after the actuating step, or both,

21. The method according to claim 19, wherein the step of actuating the stent delivery device to deploy the stent further comprises delivering gas or fluid to the stent delivery device.

22. The method according to claim 19, wherein introducing the stent delivery device further comprises introducing the guidewire prior to introducing the catheter.

23. The method according to claim 19, wherein the plurality of optical emitting libers are dispersed, about a circumference of the guidewire, catheter or the combination thereof, the optical emitting fibers directing transmitted optical radiation to the surrounding area in the lumen and collecting optical radiation back from the surrounding area of the lumen,

24. A method of deploying a stent within a lumen, comprising:
providing a stent delivery system, comprising:
a guidewire;
a catheter having a distal portion and a proximal portion;
a plurality of optical emitting fibers for measuring a surrounding area in the lumen, wherein the plurality of optical emitting fibers is located on the guidewire, the catheter, or a combination thereof;
a balloon disposed on the distal portion of the catheter; and
a stent disposed over the balloon;
utilizing the stent delivery system to place the stent at a desired position within the measured surrounding area in the lumen; and
deploying the stent within the measured surrounding area in the lumen.

25. The method according to claim 24, wherein deploying the stent further comprises:
calculating a set of optical pathlengths from returned optical radiation signals, wherein the returned optical radiation signals are measurements from between the plurality optical emitting fibers and a lumen wall;
determining a diameter of the lumen; and
expanding the stent to at least a portion of the determined diameter of the lumen.

26. The method according to claim 24, wherein, the plurality of optical emitting fibers are dispersed about a circumference of the catheter, the guidewire, or the combination thereof.
